(19) United States
(12) Patent Application Publication
(10) Pub. No.: US 2004/0191827 A1
(43) Pub. Date: Sep. 30, 2004

(54) Modulators of the hypocretin system and methods of screening therefor

(76) Inventors: Nahid S. Waleh, Palo Alto, CA (US); Thomas S. Kilduff, Menlo Park, CA (US)

Correspondence Address: REED & EBERLE LLP, 800 MENLO AVENUE, SUITE 210, MENLO PARK, CA, 94025, US

(21) Appl. No.: 10/830,638

(22) Filed: Apr. 22, 2004

Related U.S. Application Data
(63) Continuation of application No. 10/029,427, filed on Dec. 19, 2001, now abandoned.
(60) Provisional application No. 60/258,069, filed on Dec. 20, 2000.

(51) Int. Cl. C12Q 1/68; G01N 33/53; G01N 33/567

(52) U.S. Cl. 435/6; 435/7.2

(57) ABSTRACT

Methods for modulating the hypocretin system, as well as methods for identifying compounds that act as hypocretin-system modulators are provided. In modulating the hypocretin system, the method comprises administering a therapeutically effective amount of a preprohypocretin-expression modulator to an individual, wherein the preprohypocretin-expression modulator alters preprohypocretin expression in preprohypocretin-expressing cells. The method for identifying compounds comprises contacting a test compound to cells equipped with the 5′ flanking promoter of the preprohypocretin gene operably linked to a nucleic acid sequence and determining whether the test compound alters transcription of the nucleic acid sequence in the cell, wherein the test compound's ability to alter transcription is indicative of a compound that modulates the hypocretin system. The invention also provides compounds, pharmaceutical compositions, nucleic acid sequences, expression vectors, transformed host cells, and the like for carrying out the methods.

### CROSS-REFERENCE TO RELATED APPLICATION

This application is a continuation application of U.S. patent application Ser. No. 10/029,427, filed on Dec. 19, 2001, which claims priority under 35 U.S.C. §119(e)(1) to U.S. Provisional Application Serial No. 60/258,069, filed Dec. 20, 2000, all of which are incorporated by reference in their entireties herein.

### REFERENCE TO GOVERNMENT SUPPORT

This invention was funded in part by the National Institute of Mental Health under NIH Grant Nos. 1 R01MH61755 and 1 R01HL/MH59658. The United States Government has certain rights in this invention.

### TECHNICAL FIELD

The present invention relates to a method for modulating the hypocretin system in an individual, as well as to a method for identifying compounds that serve as modulators of the hypocretin system, pharmaceutical compositions containing the identified compounds, and the like. The invention has applications in the fields of neurobiology, neurochemistry, and medicine.

### BACKGROUND

Hypocretin 1 and hypocretin 2 (“Hcrt1” and “Hcrt2, respectively, or “Hcrt-1” and “Hcrt-2,” respectively; also referred to as “orexin A” and “orexin B,” respectively) are hypothalamic neuropeptides derived from a 130-amino-acid precursor molecule via proteolytic processing. See de Lecea et al. (1998), “The Hypocretins: Hypothalamus-Specific Peptides With Neuroexcitatory Activity,” Proc. Natl. Acad. Sci. USA 95:322-327; Sakurai et al. (1998), “Orexins and Orexin Receptors: A Family of Hypothalamic Neuropeptides and G-Protein-Coupled Receptors That Regulate Feeding Behavior,” Cell 92:573-585; and Kilduff et al. (2000), “The Hypocretin/Orexin Ligand-Receptor System: Implications for Sleep and Sleep Disorders,” Trends. Neurosci. 23:359-365. The precursor molecule, called “preprohypocretin,” or “prepro-orexin,” is encoded by a gene localized to human chromosome 17q21. See Sakurai et al. (1998), supra. The gene includes two exons and one intron. The first exon includes a 5′-untranslated region and a region that encodes the first seven amino acid residues of a secretory signal sequence. The second exon encodes the remaining portion of the signal sequence and preprohypocretin. One molecule of hypocretin 1 and one molecule of hypocretin 2 are produced upon proteolytic cleavage of preprohypocretin.

The hypocretins were originally identified from mRNAs of rat hypothalamus, using the directional tag polymerase chain reaction technique. See de Lecea et al. (1998), supra. Sakurai et al. describe these peptides as ligands that bind to orphan G-protein-coupled receptors and note that intracerebroventricular injections of these peptides increase food intake in rats. The two orphan receptors have been designated as hypocretin receptor 1 (also known as orexin-1 receptor, or “OX₁R”) and hypocretin receptor 2 (also known as orexin-2 receptor, or “OX₂R”). Hypocretin receptor 1 was found to be selective for hypocretin 1, whereas hypocretin receptor 2 was shown to bind both hypocretins with similar affinities. See Sakurai et al. (1998), supra. Characterized by cells having hypocretin receptors and cells expressing hypocretins, the hypocretin system has since been linked to additional biological roles such as, for example, sleep patterns, pain perception, and neuronal degeneration.

Equipped with the knowledge that the hypocretin system plays a variety of physiological roles, researchers have attempted to modulate or further characterize it with varying degrees of success. For example, WO 98/05352 (Sutcliffe et al.) and U.S. Pat. Nos. 5,969,123 to Holtzman and 6,001,963 to Bergsma et al. describe various polypeptides, as well as the polynucleotides encoding them, that are ligands for the hypocretin receptors. Some of these polypeptides are endogenous ligands for the hypocretin receptors.

Hypocretin receptor antagonists have been described in WO 00/47576 (Johns et al.) and WO 00/47577 (Coulton et al.). As discussed in WO 00/47284 (Irving et al.), administration of such antagonists provides a neuroprotective effect and has been shown to treat nausea and vomiting, irritable bowel syndrome, and other conditions associated with visceral pain.

WO 01/08720 describes the use of hypocretins and hypocretin receptors in the regulation of sleep and sleep-related disorders such as narcolepsy. Similarly, WO 01/14555 describes treatment of narcolepsy by using gene therapy to influence the expression of hypocretin receptor 1.

Although significant effort has been directed toward studying the physiology of the hypocretin system, little therapeutic success has actually been achieved to date. A partial explanation for this lack of success may lie in the use of inappropriate animal models to test proposed treatments for disorders influenced by the hypocretin system in humans. Specifically, the sleep disorder narcolepsy in humans has been associated with degeneration of the hypocretin/orexin cells, probably due to an autoimmune attack. See Thannickal et al. (2000), “Reduced Number of Hypocretin Neurons in Human Narcolepsy,” Neuron. 27:469-474. A common animal model used to test treatments for narcolepsy is that of a genetically mutated dog suffering from narcolepsy due to an exon-skipping mutation in the hypocretin receptor 2, which results in a nonfunctional hypocretin receptor 2 protein. See Lin et al. (1999), “The Sleep Disorder Canine Narcolepsy is Caused by a Mutation in the Hypocretin (Orexin) Receptor 2 Gene,” Cell 98:365-376. In contrast to humans, however, these narcoleptic dogs expressing this mutation do not undergo degeneration of the hypocretin/orexin cells. See Peyron et al. (2000), “A Mutation in a Case of Early Onset Narcolepsy and a Generalized Absence of Hypocretin Peptides in Human Narcoleptic Patients,” Nat. Med. 6:991-997 and Ripley et al. (2001), “Hypocretin Levels in Sporadic and Familial Cases of Canine Narcolepsy,” Neurobiol. Dis. 8:525-534. Thus, this particular animal model has several shortcomings in the determination of treatments for narcolepsy and other sleep disorders in humans.

The present invention provides a novel method for modulating the hypocretin system in an individual and takes into account the actual physiological and biochemical processes that control the system. Specifically, the present method modulates the hypocretin system by altering preprohypocretin expression, thereby modulating the levels of hypocretin 1 and hypocretin 2 in the individual and, consequently, affecting activation of cells bearing hypocretin receptors.

### SUMMARY OF THE INVENTION

Accordingly, it is an object of the present invention to overcome the above-mentioned disadvantages of the prior art by providing a method for modulating the hypocretin system in an individual, comprising administering a therapeutically effective amount of a preprohypocretin-expression modulator to the individual, wherein the preprohypocretin-expression modulator alters preprohypocretin expression in preprohypocretin-expressing cells.

It is another object of the invention to provide such a method wherein the modulator increases preprohypocretin expression.

It is still another object of the invention to provide such a method wherein the modulator decreases preprohypocretin expression.

It is yet another object of the invention to provide such a method wherein the modulator binds to the 5′ flanking promoter of the preprohypocretin gene.

It is a further object of the invention to provide such a method wherein the modulator is a cytokine.

It is still a further object of the invention to provide a method wherein the modulator is an interferon.

It is still yet a further object of the invention to provide a method wherein modulation of the individual's hypocretin system results in a change in the individual's sleep pattern.

Is it still another object of the invention to provide a method wherein the individual suffers from narcolepsy or other sleep/awake disorders.

It is an additional object of the invention to provide a method for identifying a compound that modulates the hypocretin system, comprising contacting a test compound to cells equipped with the 5′ flanking promoter of the preprohypocretin gene operably linked to a nucleic acid sequence and determining whether the test compound alters transcription of the nucleic acid sequence in the cells, wherein the test compound's ability to alter transcription is indicative of a compound that modulates the hypocretin system.

It is still another object of the invention to provide DNA fragments, expression vectors and host cells for carrying out the method for identifying a compound that modulates the hypocretin system.

It is yet another object of the invention to provide a compound that modulates the hypocretin system.

It is still a further object of the invention to provide a pharmaceutical composition comprising a compound that modulates the hypocretin system upon administration to an individual.

Additional objects, advantages, and novel features of the invention will be set forth in part in the description that follows, and in part, will become apparent to those skilled in the art upon examination of the following, or may be learned through routine experimentation upon practice of the invention.

In one embodiment, the invention provides a method for modulating the hypocretin system in an individual, comprising administering a therapeutically effective amount of a preprohypocretin-expression modulator to the individual, wherein the preprohypocretin-expression modulator alters preprohypocretin expression in preprohypocretin-expressing cells. As mentioned previously, the hypocretin system influences a variety of physiological processes including, for example, sleep patterns. Activation, inhibition, and maintenance of the hypocretin system are influenced by a variety of biological and chemical processes including, but not limited to, agonists and antagonists of the hypocretin receptors. The endogenous ligands for the hypocretin receptors are hypocretin 1 and hypocretin 2. Since both of these endogenous peptides are formed by proteolytic cleavage of the precursor molecule preprohypocretin, the ability to modulate or control the expression (e.g., production) of preprohypocretin by the modulators described herein represents a powerful method for modulating the hypocretin system, and, in turn, the physiological processes influenced by the hypocretin system.

Thus, the modulators described herein activate or increase the activity of the hypocretin system by increasing the expression of preprohypocretin, thereby increasing the relative amounts of hypocretin 1 and hypocretin 2 available to bind to appropriate receptors in the hypocretin system. Other modulators described herein at least partially inhibit or decrease the activity of the hypocretin system by decreasing the expression of preprohypocretin, thereby effectively decreasing the relative amounts of hypocretin 1 and hypocretin 2 available for binding. Of course, the methods described herein also include augmentation of “below-normal” activity, as well as at least partial inhibition of “above-normal” activity, of the hypocretin system.

In a preferred embodiment, for example, a method is provided for treating a narcoleptic patient, comprising administering a therapeutically effective amount of a preprohypocretin-expression modulator to the individual, wherein the preprohypocretin-expression modulator enhances preprohypocretin expression in preprohypocretin-expressing cells located in the posterior lateral hypothalamus.

In another embodiment of the invention, a method is provided to identify compounds that modulate the hypocretin system. This method comprises contacting a test compound with cells equipped with the 5′ flanking promoter of the preprohypocretin gene operably linked to a nucleic acid sequence such as a reporter and determining whether the test compound alters transcription of the nucleic acid sequence in the cell, wherein the test compound's ability to alter transcription is indicative of a compound that modulates the hypocretin system. Conveniently, this method can be carried out with naturally occurring preprohypocretin-expressing cells having the 5′ flanking promoter as exemplified by certain cells found in the posterior lateral hypothalamus or in other peripheral locations such as in bladder tissue or in the tissues comprising the gastrointestinal tract. In addition, the cells used in the method may be obtained through genetic manipulation such that cells normally lacking the 5′ flanking promoter of the preprohypocretin gene are transfected with an expression vector comprising the 5′ flanking promoter operably linked to a nucleic acid sequence coding for preprohypocretin or a different gene (e.g., reporter). In this case, transcription of the nucleic acid sequence coding for preprohypocretin or the inserted gene may be altered, thereby indicating that the compound is expected to modulate the hypocretin system upon administration to an individual via reduction of preprohypocretin expression. The invention also provides vectors and transformed cells for carrying out the screening methods.

Additional embodiments of the invention include the compounds that modulate the hypocretin system (identified by the screening method provided herein), as well as pharmaceutical compositions comprising one or more of the identified compounds. Further embodiments include DNA fragments, expression vectors, and transformed host cells used to carry out the method for identifying compounds that modulate the hypocretin system.

### BRIEF DESCRIPTION OF THE DRAWINGS

FIG. 1. is a representation of the nucleotide sequence of the 5′-flanking promoter region of the human hypocretin gene (SEQ ID NO: 1). The first residue of the transcription start site is marked as +1. The core regions of potential transcription binding sites are outlined in boxes. The sequences of polymerase chain reaction primers, as referred to in Example 1, are designated and underlined. The label with the asterisks indicates that the binding site is in the reverse orientation. Bases that deviate from the consensus sequence are marked with a dot.

FIG. 2. is a graph illustrating the effect of a deletion mutation on the promoter activity of the 5′ flanking promoter region of human hypocretin gene, as described in Example 1. Luciferase activities are shown as relative units compared to Renilla luciferase activity, which was used as an internal control plasmid. Data were derived from three different assays and are expressed as mean ±SEM.

FIG. 3. is a graph illustrating the effect of α-interferon (IFN) on the promoter activity of the 450 and 188 bp fragments of the 5′ flanking promoter region of the hypocretin gene, as described in Example 1. SY5Y cells transiently transfected with plasmids pGL3(450) and pGL3(188) were treated with 500 units/ml of α-IFN for 24 hours. Luciferase activities are shown as relative units compared to Renilla luciferase activity, which was used as an internal control plasmid. Data were derived from three different assays and are expressed as mean ±SEM.

FIG. 4. is a graph illustrating the effect of various concentrations of α-IFN on the promoter activity of the 450 bp fragment of the 5′-flanking region of hypocretin gene, as described in Example 1. SY5Y cells transiently transfected with plasmid pGL3(450) were treated with 500, 100, and 50 units/ml, respectively, of α-IFN for 24 hours. Luciferase activities are shown as relative units compared to Renilla luciferase activity, which was used as an internal control plasmid. Data were derived from three different assays and are expressed as mean±SEM.

FIG. 5. is a graph illustrating the effect of α-IFN on the promoter activity of the 450 fragment with the wild type and mutated IFN-stimulated response element (ISRE) sequence. As described in Example 1, SY5Y cells transiently transfected with the constructed plasmids were treated with 500 units/ml α-IFN for twenty-four hours. Luciferase activities are shown as relative units compared to Renilla luciferase activity, used as an internal control plasmid. Data are derived from three different assays and are expressed as mean±SEM.

### DETAILED DESCRIPTION OF THE INVENTION

Before the invention is described in detail, it is to be understood that, unless otherwise indicated, this invention is not limited to particular sequences, modulators, compounds, or processes, as such may vary. It is also to be understood that the terminology used herein is for purposes of describing particular embodiments only, and is not intended to be limiting.

As used in the specification and the appended claims, the singular forms “a,” “an,” and “the” include plural referents unless the context clearly dictates otherwise. Thus, for example, reference to a “modulator” includes a single modulator as well as a plurality of modulators, and the like.

In this specification and in the claims that follow, reference will be made to a number of terms that shall be defined to have the following meanings:

The terms “modulate” and “alter” as used herein refer to control, influence, or change of a system. Thus, “modulating the hypocretin system” entails controlling, influencing, or changing the preexisting status of the hypocretin system. Similarly, a “preprohypocretin-expression modulator” refers to a substance that can control, influence, or change the expression of preprohypocretin from an initial or baseline level. Also, a modulator “alters preprohypocretin expression” when it controls, influences, or changes the expression of preprohypocretin when compared to the expression of preprohypocretin without the benefit of the modulator. Examples of control, influence, or change in any given system or for any given action encompass the ability to enhance, decrease, inhibit, restore, preserve, or maintain the particular system or action.

As used herein, the term “hypocretin system” refers to the totality of the cells, receptors, transmitters, ligands, nucleic acid sequences, projections, processes, actions, and functions of hypocretins and their corresponding receptors. Although the hypocretin system may be modulated in a number of different ways, including increasing the expression of hypocretin itself, the present invention is directed to modulation of the hypocretin system via altering the expression of the hypocretin precursor, preprohypocretin.

As used herein, the term “nucleic acid sequence” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. In addition, the terms refer to single-stranded and double-stranded forms of these polymers. Thus, these terms include known types of nucleic acid sequence modifications, for example, substitution of one or more of the naturally occurring nucleotides with an analog; internucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters, etc.); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochemistry 9:4022, 1970).

The terms “expression vector” or “vector” refer to a compound and/or composition that transduces, transforms, or infects a host cell, thereby causing the cell to express nucleic acids and/or proteins. An “expression vector” contains a sequence of nucleic acids (ordinarily RNA or DNA) to be expressed by the host cell. Optionally, the expression vector also comprises materials to aid in achieving entry of the nucleic acid into the host cell, such as a virus, liposome, protein coating, or the like. The expression vectors contemplated for use in accordance with the present invention include those into which a nucleic acid sequence can be inserted, along with any preferred or required operational elements. Further, the expression vector must be one that can be transferred into a host cell and replicated therein. Preferred expression vectors are plasmids, particularly those with restriction sites that have been well documented and that contain the operational elements preferred or required for transcription of the nucleic acid sequence. Such plasmids, as well as other expression vectors, are well known to those of ordinary skill in the art.

The term “transduce” as used herein refers to the transfer of a sequence of nucleic acids into a host cell. Only when the sequence of nucleic acids becomes stably replicated by the cell does it become “transformed.” As will be appreciated by those of ordinary skill in the art, “transformation” may take place either by incorporation of the sequence of nucleic acids into the cellular genome, i.e., chromosomal integration, or by extrachromosomal integration. In contrast, an expression vector, e.g., a virus, is “infective” when it transduces a host cell, replicates, and (without the benefit of any complementary virus or vector) spreads progeny expression vectors, e.g., viruses, of the same type as the original transducing expression vector to other microorganisms, wherein the progeny expression vectors possess the same ability to reproduce. The host cell that is transformed may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus. A preferred prokaryotic cell is Escherichia coli. Preferred eukaryotic cells are those derived from fungal, insect, or mammalian cell lines.

The term “operably linked” refers to a functional linkage between a nucleic acid expression control sequence (such as a promoter) and a second nucleic acid sequence, wherein the expression control sequence directs transcription of the nucleic acid corresponding to the second sequence.

The terms “modulator,” “active agent,” “a compound that modulates the hypocretin system,” and variations thereof are used herein to refer to a chemical compound that induces a desired pharmacological or physiological effect, i.e., in this case, modulation of the hypocretin system. The primary active agents herein are hypocretin system modulators. The terms also encompass pharmaceutically acceptable, pharmacologically active derivatives of those active agents specifically mentioned herein, including, but not limited to, salts, esters, amides, prodrugs, active metabolites, conjugates (e.g., conjugates of polyethylene glycol), analogs, and the like. When the terms “modulator,” “active agent,” “a compound that modulates the hypocretin system,” and variations thereof are used, or when a modulator such as a cytokine is specifically identified, it is to be understood that applicants intend to include the active agent per se as well as pharmaceutically acceptable, pharmacologically active salts, esters, amides, prodrugs, metabolites, conjugates, analogs, etc.

By “pharmaceutically acceptable,” such as in the recitation of a “pharmaceutically acceptable carrier,” or a “pharmaceutically acceptable acid addition salt,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”) as in a “pharmacologically active” derivative or metabolite, refers to a derivative or metabolite having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well, i.e., therapeutically effective as a modulator of the hypocretin system.

“Carriers” or “vehicles” as used herein refer to conventional pharmaceutically acceptable carrier materials suitable for drug administration, and include any such materials known in the art that are nontoxic and do not interact with other components of a pharmaceutical composition or drug delivery system in a deleterious manner.

The terms “treating” and “treatment” as used herein refer to the ability to prevent, cure, ameliorate, and/or alleviate the symptoms and/or the underlying cause of a disease, disorder, or condition. In treating an individual patient, the individual is preferably mammalian, such as a human, although other species, e.g., dogs, are included as well. Patients of particular interest are those that suffer from sleeping disorders, which include, by way of illustration and not limitation, narcolepsy, insomnia, age-related sleep disorders, sleep disorders due to jet-lag, and so forth.

By an “effective” amount or a “therapeutically effective amount” of a drug or pharmacologically active agent is meant a nontoxic but sufficient amount of the drug or agent to provide the desired effect. The amount that is “effective” will vary from subject to subject, depending on the age and general condition of the individual, the particular modulator or modulators, and the like. Thus, it is not always possible to specify an exact “effective amount.” However, an appropriate “effective” amount in any individual case may be determined by one of ordinary skill in the art using routine experimentation.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

In a first embodiment, the invention provides a method for modulating the hypocretin system. As described previously, the hypocretin system plays a role in a large number of physiological processes. Although hypocretin expression is limited to a relatively small population of cells, projections of hypocretin-expressing cells are widely distributed in the brain and spinal cord, with particularly abundant processes located throughout the hypothalamus. While not wishing to be bound by theory, the distribution and arrangement of the hypocretin system is believed to account for its wide array of physiological roles.

Modulation of the hypocretin system in an individual is effected by administering a preprohypocretin-expression modulator. The modulator alters preprohypocretin expression by a preprohypocretin-expressing cell, for example, by enhancing or decreasing such expression. The preprohypocretin-expressing cells are preferably located in the posterior lateral hypothalamus or in peripheral tissue (i.e., non-central nervous system tissue) such as the tissues comprising the gastrointestinal tract and bladder tissue. As described previously, preprohypocretin is the precursor molecule that, upon proteolytic cleavage, results in the release of a single molecule of hypocretin 1 and a single molecule of hypocretin 2. Thus, a modulator that enhances preprohypocretin expression effectively increases the hypocretin levels, thereby activating the hypocretin system through agonist activity on hypocretin receptors. In contrast, a modulator that decreases preprohypocretin expression has the opposite result, i.e., a modulator that decreases preprohypocretin expression effectively decreases total hypocretin levels, thereby deactivating or down regulating the hypocretin system via a relative lack of available endogenous agonists for hypocretin receptors.

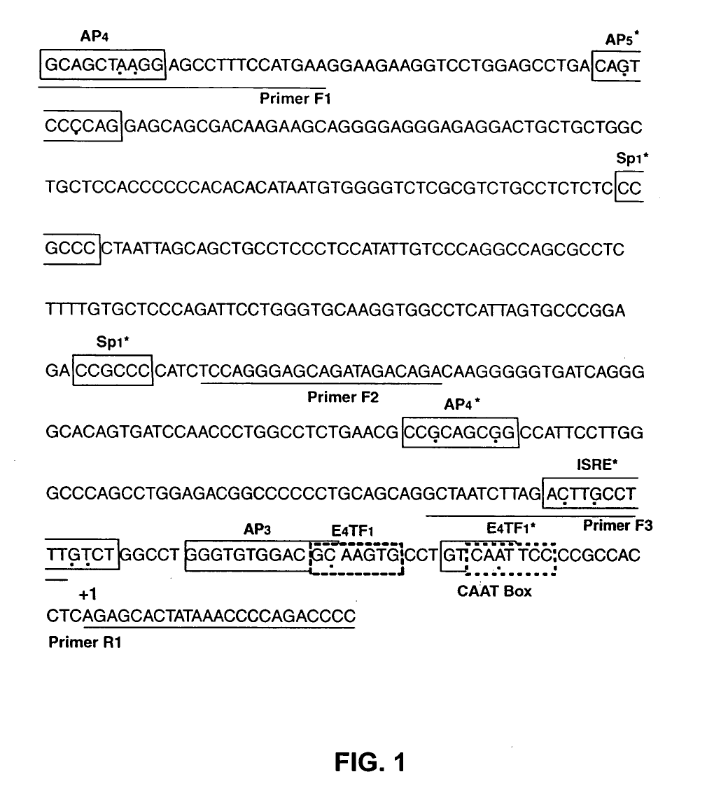

Although the invention is not limited by the method through which the modulator alters preprohypocretin expression, in a preferred embodiment the modulator binds to the 5′ flanking promoter of the preprohypocretin gene. The sequence for this gene has been assigned SEQ ID NO: 1 and is provided in FIG. 1. As will be appreciated by one of ordinary skill in the art, some binding events enhance gene expression while other binding events decrease gene expression. Consequently, the modulators described herein preferably bind to a portion of the 5′ flanking promoter of the preprohypocretin gene, irrespective of whether the modulator enhances or decreases preprohypocretin expression.

Particularly preferred modulators that act to decrease preprohypocretin expression include the class of agents known as immunomodulators. Among known immunomodulators, cytokines are particularly preferred. Some cytokines, e.g., interferons, will decrease preprohypocretin expression, while other cytokines will enhance preprohypocretin expression. Particularly preferred cytokines that act to decrease preprohypocretin expression are the interferons. Although any interferon may be administered, alpha-interferon, beta-interferon, gamma-interferon, and combinations thereof are preferred. A particularly preferred interferon is alpha-interferon.

The modulator, either alone or with one or more additional active agents, may be administered; or a combination of modulators, optionally with one or more additional active agents, may be administered. The modulator(s) and optional additional active agent(s) may be present in either a single formulation or in separate formulations, and in the latter case, may be administered either simultaneously or sequentially. The additional active agent will generally, although not necessarily, be one that is effective in treating the same condition, disease, or disorder for which the modulator is given. For example, if the modulator is administered to decrease the individual's desire for sleep, one or more wakefulness-promoting drugs may be given as additional active agents. Such wakefulness-promoting drugs, e.g., amphetamine, amphetamine homologues, caffeine, cathinone, cocaine, ephedrine, methamphetamine, methylphenidate, modafinil, pemoline, phenmetrazine, and combinations thereof, as well as additional active agents for treating other conditions, diseases, or disorders, are known to those of ordinary skill in the art.

Preferred additional active agents are selected from the group of wakefulness-promoting drugs, tricyclic antidepressants, tetracyclic antidepressants, selective serotonin reuptake inhibitors, monoamine oxidase inhibitors, and combinations thereof. Specific examples of additional active agents include, without limitation, modafinil, amphetamine, amphetamine homologues, caffeine, cocaine, cathinone, ephedrine, theophylline, theobromine, methylphenidate, dextroamphetamine, methamphetamine, pemoline, phenmetrazine, mazindol, selegiline, ritanserin, violoxazine, CRL40476, clomipramine, imipramine, desipramine, fluoxetine, paroxitine, sertraline, gammahydroxybutyrate, clonazepam, carbamazepine, yohimbine, and combinations thereof.

Any of the modulators and active agents described herein may be administered in the form of a salt, ester, amide, prodrug, active metabolite, conjugate, derivative, or the like, provided that the salt, ester, amide, prodrug, metabolite, conjugate, or other derivative is suitable pharmacologically, i.e., effective in the present method. Salts, esters, amides, prodrugs, conjugates, and other derivatives of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). For example, acid addition salts may be prepared from a free base (e.g., a compound containing a primary amino group) using conventional methodology involving reaction of the free base with an acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Conversely, preparation of basic salts of any acidic moieties that may be present may be carried out in a similar manner using a pharmaceutically acceptable base, such as sodium hydroxide, potassium hydroxide, ammonium hydroxide, calcium hydroxide, trimethylamine, or the like. Preparation of esters involves reaction of a hydroxyl group with an esterification reagent, such as an acid chloride, or esterification of a free carboxylic acid group. Amides may be prepared from esters using suitable amine reactants, or they may be prepared from an anhydride or an acid chloride by reaction with ammonia or a lower alkyl amine. Prodrugs, conjugates, and active metabolites may also be prepared using techniques known to those skilled in the art or described in the pertinent literature. Prodrugs and conjugates are typically prepared by covalent attachment of a moiety that results in a compound that is therapeutically inactive until modified by an individual's metabolic system.

In addition, many of the active agents contain chiral centers and can thus be in the form of a single isomer or a racemic mixture of isomers. Chiral active agents may be in isomerically pure form, or they may be administered as a racemic mixture of isomers.

Other derivatives and analogs of the active agents may be prepared using standard techniques known to those skilled in the art of synthetic organic chemistry, or may be deduced by reference to the pertinent literature.

The modulators and active agents of the invention may be administered orally, parenterally (e.g., via subcutaneous, intravenous, or intramuscular injection), rectally, buccally, sublingually, nasally, by inhalation, topically, transdermally, intracerebralventricularly, or via an implanted reservoir in dosage forms containing conventional nontoxic pharmaceutically acceptable carriers and excipients. The amount of the compound administered will, of course, be dependent on the particular active agent, the condition or disorder being treated, the severity of the condition or disorder, the subject's weight, the mode of administration, and other pertinent factors known to the prescribing physician or health care professional. Generally, however, the dosage will be in the range of approximately 0.001 mg/kg/day to 100 mg/kg/day, more preferably in the range of about 0.1 mg/kg/day to 10 mg/kg/day.

Suitable compositions and dosage forms include tablets, capsules, caplets, gel caps, troches, dispersions, suspensions, solutions, syrups, transdermal patches, gels, powders, magmas, lozenges, creams, pastes, plasters, lotions, discs, suppositories, liquid sprays for nasal or oral administration, dry powder or aerosolized formulations for inhalation, and the like.

Oral dosage forms are preferred for those modulators that are orally active, and include tablets, capsules, caplets, solutions, suspensions, and/or syrups, and may also comprise a plurality of granules, beads, powders, or pellets that may or may not be encapsulated. Such dosage forms are prepared using conventional methods known to those in the field of pharmaceutical formulation and described in the pertinent texts, e.g., in Remington: The Science and Practice of Pharmacy, 20th Edition, Gennaro, A. R., Ed. (Lippincott, Williams and Wilkins, 2000). Tablets and capsules represent the most convenient oral dosage forms, in which case solid pharmaceutical carriers are employed.

Depending on the intended mode of administration, the pharmaceutical formulation may be a solid, semisolid, or liquid (such as, for example, a tablet, a capsule, caplets, a liquid, a suspension, an emulsion, a suppository, granules, pellets, beads, a powder, or the like), preferably in unit dosage form suitable for single administration of a precise dosage. Suitable pharmaceutical compositions and dosage forms may be prepared using conventional methods known to those in the field of pharmaceutical formulation and described in the pertinent texts and literature, e.g., in Remington: The Science and Practice of Pharmacy, 19th Ed. (Easton, Pa.: Mack Publishing Co., 1995).

Tablets may be manufactured using standard tablet processing procedures and equipment. Direct compression and granulation techniques are preferred. In addition to the active agent, tablets will generally contain inactive, pharmaceutically acceptable carrier materials, such as binders, lubricants, disintegrants, fillers, stabilizers, surfactants, coloring agents, and the like. Binders are used to impart cohesive qualities to a tablet, and thus ensure that the tablet remains intact. Suitable binder materials include, but are not limited to, starch (including corn starch and pregelatinized starch), gelatin, sugars (including sucrose, glucose, dextrose, and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methylcellulose, microcrystalline cellulose, ethyl cellulose, hydroxyethyl cellulose, and the like), and Veegum®. Lubricants are used to facilitate tablet manufacture, promoting powder flow and preventing particle capping (i.e., particle breakage) when pressure is relieved. Useful lubricants are magnesium stearate, calcium stearate, and stearic acid. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard drug decomposition reactions that include, by way of example, oxidative reactions.

Capsules are also preferred oral dosage forms, in which case the active agent-containing composition may be encapsulated in the form of a liquid or solid (including particulates such as granules, beads, powders, or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, Nineteenth Edition. (1995) cited supra, which describes materials and methods for preparing encapsulated pharmaceuticals.

Preparations according to this invention for parenteral administration include sterile nonaqueous solutions, suspensions, and emulsions. Examples of nonaqueous solvents or vehicles are propylene glycol, polyethylene glycol, vegetable oils (including olive oil and corn oil), gelatin, and injectable organic esters, such as ethyl oleate. Parenteral formulations may also contain adjuvants, such as preserving, wetting, emulsifying, and dispersing agents. The formulations are rendered sterile by incorporation of a sterilizing agent, filtration through a bacteria-retaining filter, irradiation, or heat. They can also be manufactured using a sterile injectable medium.

The modulator may also be administered through the skin or mucosal tissue using conventional transdermal drug delivery systems, wherein the modulator is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the drug-containing reservoir and skin contact adhesive are present as separate and distinct layers, with the adhesive underlying the reservoir. In this case, the reservoir may be either a polymeric matrix (as described above) or a liquid or hydrogel reservoir; or it may take some other form. Transdermal drug delivery systems may, in addition, contain a skin permeation enhancer.

The modulators may also be administered intranasally or by inhalation. Compositions for nasal administration are generally liquid formulations for administration as a spray or in the form of drops, although powder formulations for intranasal administration, e.g., insufflations, are also known.

Formulations for inhalation may be prepared as an aerosol, either a solution aerosol, in which the modulator is solubilized in a carrier (e.g., propellant), or a dispersion aerosol, in which the modulator is suspended or dispersed throughout a carrier and an optional solvent. Nonaerosol formulations for inhalation may take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosa, 1 and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate, and combinations thereof), and/or a suspending agent (e.g., agar, bentonite, microcrystalline cellulose, sodium carboxymethylcellulose, hydroxypropyl methylcellulose, tragacanth, Veegum®, and combinations thereof). Nonaerosol formulations for inhalation may also comprise dry powder formulations, particularly insufflations, in which the powder has an average particle size of about 0.1 μm to 50 μm, preferably about 1 μm to 25 μm.

The inventive method of modulating the hypocretin system finds uses in a variety of contexts, and the present method is not limited with respect to any particular application. A preferred application, however, is modulation of the hypocretin system to provide a change in the individual's sleep pattern. As will be recognized by one of ordinary skill in the art, a change in sleep pattern encompasses not only the duration of sleep, but also the quality and type (e.g., REM, deep, etc.). The ability to alter sleep patterns is advantageous, not only to individuals suffering from a sleep disorder, but also to those individuals who desire to maintain wakefulness (e.g., military personnel on special missions or individuals piloting planes or ships for extended periods of time). A modulator that enhances the expression of preprohypocretin in an individual will decrease the individual's desire for sleep, and thereby, help to maintain wakefulness. Such modulators are particularly useful in treating individuals who suffer from 1+−. In addition, a modulator that decreases expression of preprohypocretin will increase an individual's desire for sleep, and thereby, have value in treating individuals who suffer from insomnia.

Modulation of the hypocretin system is also useful in the treatment of individuals who suffer from mood disorders, chronic fatigue syndrome, or an attention deficit disorder. WO 01/08720 suggests modulation of the hypocretin system to alleviate these conditions, however, the reference does not describe inhibiting modulation of the hypocretin system by controlling expression of preprohypocretin.

Modulating the hypocretin system by decreasing the expression of preprohypocretin may be useful in treating individuals who suffer from neuronal degeneration resulting from prior ischemic events. Similarly, decreased expression of preprohypocretin through administration of an appropriate modulator may alleviate nausea and vomiting, as well as irritable bowel syndrome and visceral pain, e.g., pain associated with migraine, angina, urge-type incontenence, and so forth. Each of these conditions is described as being preventable or treatable with administration of a receptor antagonist in WO 00/47284. In contrast, the method described herein provides for the prevention and/or treatment of these conditions through administration of a modulator that decreases preprohypocretin expression.

Administering a modulator of preprohypocretin expression may also have beneficial effects on bladder function. Consequently, depending on the type of modulator administered, an individual may be treated for urinary incontinence or may be provided diuretic therapy. As it has been suggested that hypocretins modulate contractility in the bladder (Morgan et al., “Evidence of Hypocretins in the Urinary Bladder,” abstract of presentation at the annual meeting of the Society for Neuroscience, Nov, 14, 2001), the current method of controlling expression of preprohypocretin may be used to modulate the activity of the bladder.

As hypocretins play a significant role in the regulation of food intake, the present modulators may be used to treat an individual suffering from an eating disorder. Thus, for example, administering a modulator that enhanced preprohypocretin expression would likely result in increased appetite. Such a result would benefit, without limitation, cancer patients who have lost their appetite due to chemotherapy treatment. In addition, administering a modulator that decreased preprohypocretin expression would result in loss of appetite, thereby providing a potential therapy for individuals suffering from obesity.

Although modulators of preprohypocretin, e.g., cytokines, have been identified herein, additional modulators may be available. Consequently, the invention also provides a method for identifying compounds that modulate the hypocretin system, comprising contacting a test compound to cells equipped with the 5′ flanking promoter of the preprohypocretin gene operably linked to a nucleic acid sequence and determining whether the test compound alters transcription of the nucleic acid sequence in the cell. The test compound's ability to alter transcription is indicative of a compound that modulates the hypocretin system.

Naturally occurring cells that express preprohypocretin endogenously, such as those located in the posterior lateral hypothalamus, bladder tissue or tissue of the gastrointestinal tract, may be useful in carrying out compound-screening methods. Such cells, however, may be difficult to obtain. Consequently, the invention also provides for cells genetically manipulated to be equipped with the 5′ flanking promoter of the preprohypocretin gene.

Any prokaryotic or eukaryotic host cell may be employed in the present method as long as it remains viable after being transformed with a sequence of nucleic acids. Preferred cells include, without limitation, neuroblastoma cells, kidney cells, and ovary cells. Preferably, the host cell would not be adversely affected by the transduction of the necessary nucleic acid sequences.

Sequences of nucleic acids coding for the 5′ flanking promoter of the preprohypocretin gene are prepared by any suitable method known to those of ordinary skill in the art, including, for example, direct chemical synthesis or cloning. For direct chemical synthesis, formation of a polymer of nucleic acids typically involves sequential addition of 3′-blocked and 5′-blocked nucleotide monomers to the terminal 5′-hydroxyl group of a growing nucleotide chain, wherein each addition is effected by nucleophilic attack of the terminal 5′-hydroxyl group of the growing chain on the 3′-position of the added monomer, which is typically a phosphorus derivative (such as a phosphotriester, phosphoramidite, or the like). Such methodology is known to those of ordinary skill in the art and is described in the pertinent texts and literature (e.g., in D. M. Matteuci et al. (1980), Tet. Lett. 521:719; U.S. Pat. No. 4,500,707 to Caruthers et al.; and U.S. Pat. Nos. 5,436,327 and 5,700,637 to Southern et al.). In addition, the desired sequences may be isolated from natural sources by splitting DNA using appropriate restriction enzymes, separating the fragments using gel electrophoresis, and thereafter, recovering the desired nucleic acid sequence from the gel via techniques known to those of ordinary skill in the art, such as utilization of polymerase chain reactions.

The nucleic acid sequence coding for the 5′ flanking promoter of the preprohypocretin gene must be operatively linked with a second nucleic acid sequence. The second nucleic acid sequence may code for preprohypocretin or a different gene. Both the 5′ flanking promoter and the second nucleic acid sequence are then incorporated in an appropriate expression vector.

The invention is not limited with respect to the process by which the nucleic acid sequence is incorporated into the expression vector. Those of ordinary skill in the art are familiar with the necessary steps for incorporating nucleic acid sequences into an expression vector. A typical expression vector contains the nucleic acid sequence for a particular gene preceded by one or more regulatory regions (i.e., the 5′ flanking promoter of the preprohypocretin gene), along with a ribosome binding site.

Although any suitable expression vector may be used to incorporate the desired sequences, readily available expression vectors include, without limitation, plasmids, such as the pGL3-basic plasmid. Of course, such expression vectors may only be suitable for a particular host cell. One of ordinary skill in the art, however, can readily determine through routine experimentation whether any particular expression vector is suited for any given host cell. For example, the expression vector can be introduced into the host cell, which is then monitored for viability and expression of the sequence(s) contained in the vector. In addition, reference may be made to the relevant texts and literature, which describe expression vectors and their suitability to any particular host cell.

The expression vectors of the invention must be introduced or transferred into the host microorganism. Such methods for transferring the expression vectors into host microorganisms are well known to those of ordinary skill in the art. One method involves placing the host cell in a transfection medium for a suitable time period, e.g., about five hours to about 24 hours. Suitable transfection media are known to those of ordinary skill in the art. Also, microinjection of the nucleic acid sequence(s) provides the ability to transfect a host cell. Other transfection means, such as lipid complexes, liposomes, and dendrimers, may also be employed. Those of ordinary skill in the art can transfect a host microorganism with a desired sequence using these or other methods.

A variety of methods are available for identifying a transfected host microorganism. For example, using a suitable dilution, a culture of potentially transfected host cells may be grown and tested for expression of the desired nucleic acid sequence. This method includes testing for luciferase activities when luciferase plasmids are employed. Such a method is conveniently carried out using commercially available systems, such as the Dual-Luciferase® reporter system (Promega, Madison, Wis.).

Once the host cell has been transformed with the expression vector, the host cell is allowed to grow. This process typically entails culturing the cells in a suitable medium. As the host cells grow and/or multiply, transcription and ultimate expression of an operatively linked gene (e.g., reporter) to the 5′ flanking promoter are effected.

Alteration of transcription in the presence of the test compound (as compared with transcription in the absence of the test compound) can take place by conducting the assay simultaneously, or by conducting prior experiments to establish typical transcription levels without addition of the test compound. Transcription levels can be determined directly by assaying mRNA using, for example, complementary oligonucleotide probes. In addition, transcription levels can be determined using expression of the operably linked gene as a surrogate. Conveniently a “reporter” gene may be used such that, once expressed, the “reporter” gene offers facile detection. Examples of reporters include, without limitation, luciferase, LacZ, and beta-galactosidase. Use of probes, genes and reporters in detecting transcription both quantitatively and qualitatively are known to those of ordinary skill in the art.

In carrying out the method for identifying a modulator for the hypocretin system, the test compound may enhance or decrease transcription levels, thereby enhancing or decreasing, respectively, expression of the operably linked gene. Preferred compounds will bind to the 5′-flanking promoter of the preprohypocretin gene. Conveniently, the method may be carried out in vitro.

In carrying out the present method, several genetic tools can be prepared. Initially, an isolated DNA fragment coding for the 5′ flanking promoter of the preprohypocretin gene, preferably comprising the nucleotide sequence of SEQ ID NO: 1, is synthesized. This fragment can then be conveniently inserted into an expression vector. Once the expression vector is transfected into an appropriate host cell, the host cell transformed thereby can be conveniently cultured to supply cells sufficient for a plurality of screenings.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

### EXPERIMENTAL

Human neuroblastoma cells (SY5Y), African monkey kidney cells (COS-7), mouse neuroblastoma cells (NS20Y), and Chinese hamster ovary cells (CHO) were obtained from the cell culture collection using cell lines originally purchased from the American Type Culture Collection (Manassas, Va.). All cells were grown as monolayers in Dulbecco's modified Eagle's medium supplemented with 10% fetal calf serum.

Cells were plated at 5×10⁹ cells per well in six-well plates. At 24 hours, the medium was removed and cells were washed once with serum-free medium. Cells were transfected with 2 μg test Luciferase plasmid, 1 μg Renilla plasmid (an internal control to adjust for transfection efficiency), and 6 μg of Lipofectamine™ 2000 reagent (Gibco BRL, Gaithersburg, Md.) in 1 ml of serum-free medium. Following an incubation period of five hours for the SY5Y cells and 24 hours for the other cells, the transfection medium was removed and replaced with the growth medium. Cells were allowed to recover in the growth medium for 24 hours before any treatment.

A series of expression plasmids was constructed. Briefly, the polymerase chain reaction (PCR) was used to clone the 5′-flanking region of the human hypocretin gene. The PCR-generated fragments were obtained by using a single reverse primer and three different forward primers. The reverse primer (R−1),

5′-GGGGTCTGGGGTTTATAGTGCTCT-3′ (SEQ ID NO: 2),

was complementary to the start of exon 1 of the hypocretin gene (Sakurai et. al. (1999), “Structure and Function of Human Prepro-Oxerin Gene,” J. Biol. Chem. 274:17771-17776). The forward primers to generate the 450, 188, and 69 bp PCR fragments were:

| | |
|---|---|
| (F-1) | |
| 5′-GCAGCTAAGGAGCCTTTCCATGAA-3′; | (SEQ ID NO: 3) |
| (F-2) | |
| 5′-TCCAGGGAGCAGATAGACAGA-3′; and | (SEQ ID NO: 4) |
| (F-3) | |
| 5′-GCTAATCTTAGACTTGCCTTT-3′, | (SEQ ID NO: 5) |

respectively. For generation of the 450 bp fragment, human DNA was used as the template. The 188 and 69 bp fragments were generated using the cloned 450 bp sequence as the template. The PCR products were first cloned into pPCR-Script™ Amp SK(+) plasmid (Stratagene, La Jolla, Calif.) for sequencing before they were subcloned into pGL3-Basic, a promoter-less luciferase expression plasmid (Promega, Madison, Wis.), for the expression studies. The plasmids were designated pGL3(450), pGL3(188), and pGL3(69). The luciferase activities of transfected cells were assayed on a TD-20/20 Luminometer (Turner Designs, Sunnyvale, Calif.) using a Dual-Luciferase™ Reporter system (Promega, Madison, Wis.), which expresses luciferase activity as relative units of activity of the firefly and Renilla luciferases.

A special-purpose mutant interferon (IFN)-stimulated response element (ISRE) was constructed. The 450 bp 5′-flanking region of the human hypocretin gene (hcrt) with a mutant IFN-stimulated response element (ISRE) was generated by PCR. In the first step, two separate PCRs were carried out using the wild type 450 bp as the template. In one PCR, the F−1 primer was used with a reverse primer 5′-CTACGCGCTCGGGGCTAAGATTAGCCTGCT-3′ (SEQ ID NO: 6), and in the second PCR, a forward primer:

5′-GCCCCGAGCGCGTAGGGCCTGGGTGTGG-3′ (SEQ ID NO: 7)

was used with R-1. In the final PCR, primers F−1 and R−1 were used with equal amounts of the products of the first two PCRs as the template. The PCR product was first cloned into pPCR-Script™ Amp SK(+) plasmid (Stratagene, La Jolla, Calif.) for sequencing before it was subcloned into pGL3-Basic for the expression studies.

The cells were transfected with the appropriate expression plasmid as described above. After recovery in growth medium for 24 hours, they were treated with different concentrations of α-IFN (Biosource International, Camarillo, Calif.) in 1 ml of growth medium. After overnight incubation, cells were lysed, and the lysates were assayed for luciferase activity.

The PCR amplification of human genomic DNA with primers F−1 and R−1 gave a fragment of 474 bp. This fragment contained 450 bp of the 5′-flanking sequence and 24 bp of the start of the hypocretin transcript (see Sakurai et al. (1999), supra). DNA sequencing showed that this fragment had a sequence identical to the sequence published previously (see Sakurai et al. (1999), supra). To investigate whether the cloned fragment had promoter activity, the expression plasmid pGL3(450) was transiently transfected into SY5Y, COS-7, NS20Y, and CHO cells. The results showed that the 450 bp fragment had the ability to promote luciferase expression in all four cell lines. The expression levels varied in different cell lines and ranged between two- and 20-fold over the background of pGL3-Basic. Since the best expression was obtained with SY5Y cells, this cell line was selected for further studies.

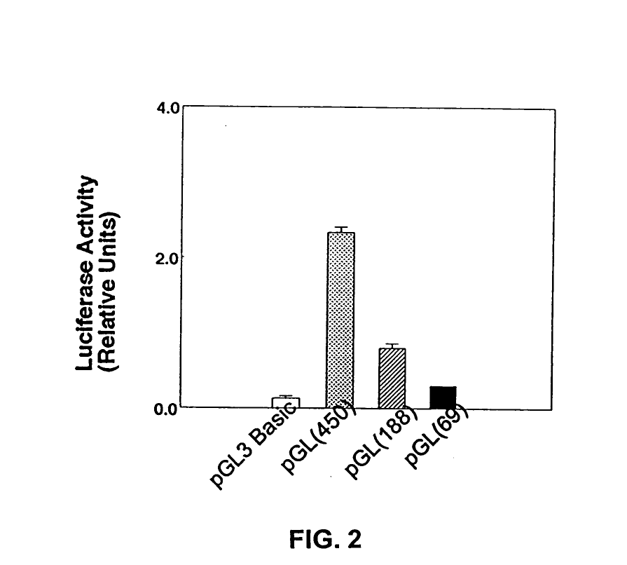

DNA sequence analysis by MacVector 6.5 (Oxford Molecular, Oxford, UK) showed that the 450 bp upstream regulatory region of human hypocretin contained multiple transcription factor binding sites, including two AP4, two Sp1, two E4TF1, one AP3, and one AP5 site; an ISRE; and a CAAT box (FIG. 1). To localize the region essential for the observed promoter activity, 5′ deletions were generated and transiently expressed in SY5Y cells. The two Sp1 sites, one of the two AP4 sites, and the only AP5 site were deleted to generate the 188 bp promoter. The second AP4 site was additionally deleted to construct the 69 bp fragment. As shown in FIG. 2, the plasmid pGL3(188) with a 262 bp deletion had about 50% of the pGL3(450) activity, and plasmid pGL3(69) with a 381 bp deletion showed almost no activity. The 450 bp promoter sequence lacked a putative TATA box near the initiation site. Although there is a TATAAA sequence in position 5-10, this sequence is apparently nonfunctional (see Sakurai et al. (1999), supra).

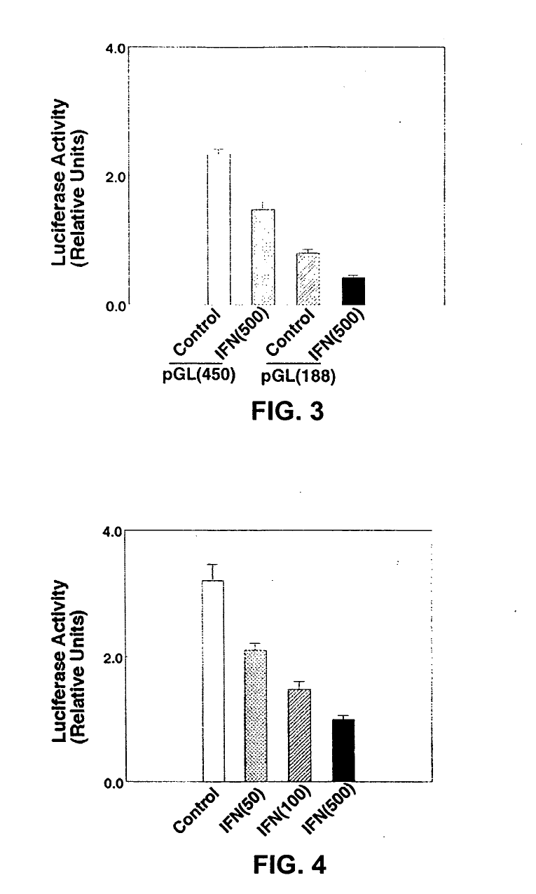

The region −45 to −58 upstream of the reported transcription start site (see Sakurai et al. (1999), supra) includes an ISRE (FIG. 1). To determine whether this ISRE was sufficient to confer an effect by α-IFN, pGL3(450) and pGL3(188) expression plasmids were transiently transfected into SY5Y cells. After treatment with α-IFN (500 U/ml) for 24 hours, a significant (i.e., 50-70%) reduction in luciferase activity was observed with both plasmids, showing that this element can mediate a response to α-IFN (FIG. 3). The inhibition of luciferase activity was not due to the antiproliferative effect of α-IFN, since the activity of the control Renilla plasmid was unchanged in all the cytokine experiments. Treatment with different concentrations of α-IFN demonstrated that the cytokine response in the cells transfected with pGL3(450) plasmid was dose-dependent (FIG. 4).

To demonstrate that the inhibitory effect of α-IFN was mediated through the ISRE, the sequence was mutated in the 450 bp PCR fragment from

| | |
|---|---|
| 5′-ACTTGCCTTTGTCT-3′ | (SEQ ID NO: 8) |
| 5′-CCCCGAGCGCGTAG-3′. | (SEQ ID NO: 9) |

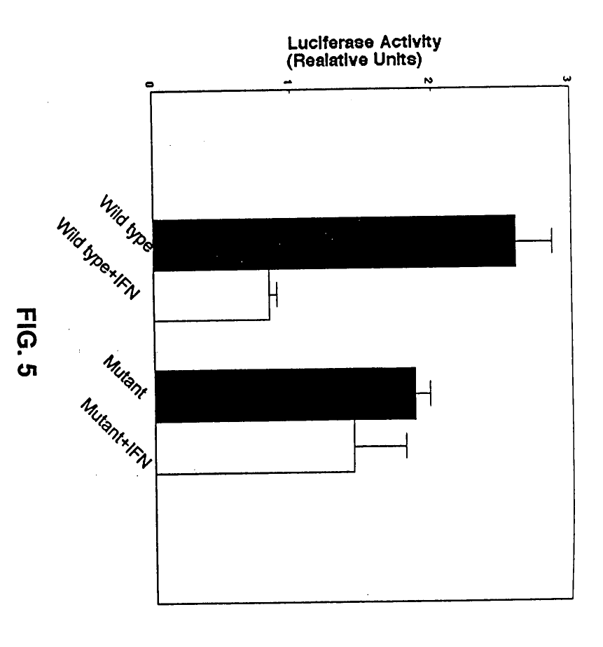

As shown in FIG. 5, the 450 bp fragment with the mutated ISRE had promoter activity, and this activity was not inhibited by treatment with α-IFN.

Thus, the 450 bp from the 5′-flanking region of the hypocretin gene is sufficient to promote gene expression in vitro. Deletion of 262 bp at the 5′-end (which included the two Sp-1 sites, one of the two AP4 sites, and the only AP5 binding site) resulted in an approximately 50% reduction in promoter activity. Sp1, a zinc finger protein, is known to upregulate transcription by binding the GC box motifs of the promoters of a variety of mammalian and viral genes. See Kadonaga et al. (1987), “Isolation of cDNA Encoding Transcription Factor Sp1 and Functional Analysis of the DNA Binding Domain,” Cell 51:1079-1090. AP4 and AP5, which are also zinc finger proteins, interact with specific promoter sequences as well to enhance gene expression. See Hu et al. (1990), “Transcription Factor AP-4 Contains Multiple Dimerization Domains That Regulate Dimer Specificity,” Genes Dev. 4:1741-1752 and Hou et al. (1995), “AP-4 and AP-5 Like Proteins From Mouse L Cells Are Trans-Activators and Bind to the GT-II Region of SV40 Early TRE in a Mutually Exclusive Manner,” Gene 162:197-203.

Since the initial 69 bp of the flanking region of the hypocretin gene did not confer any luciferase activity, the two E4TF1 sites located at positions −11 to −17 and −23 to −29 are either not functional or they require the full-length 450 bp promoter sequence for activity. E4TF1, which is a member of the Ets transcription factor family (Watanabe et al. (1993), “CDNA Cloning of Transcription Factor E4TF1 Subunits with ETS and Notch Motifs,” Mol. Cell. Biol. 13:1385-1391), is essential for the core promoter activity of certain genes. See Sowa et al. (1997), “Retinoblastoma Binding Factor I Site in the Core Promoter Region of the Human RB Gene is Activated by hGABP/E4TF1,” Cancer Res. 57:3145-3148; Kamura et al. (1997), “Characterization of the Human Thrombopoietin Gene Promoter,” J. Biol. Chem. 272:11361-11368; and Vassias et al. (1998), “Regulation of Human B19 Parvovirus Promoter Expression by hGABP (E4TF1) Transcription Factor,” J. Biol. Chem. 273:8287-8293. It is also possible that sequences outside (perhaps flanking) the 450 bp region may be required for activity of the 69 bp fragment.

Because of the presence of the ISRE, both the pGL(450) and pGL(188) reporter constructs were treated with α-IFN, which reduced the luciferase activity of both plasmids. Treatment with α-IFN down-regulated the expression of the pGL(450) luciferase reporter in a dose-dependent manner. This finding is consistent with the published physiological studies that indicate α-IFN is a sleep-promoting cytokine (see Krueger et al. (1994), “Microbial Products and Cytokines in Sleep and Fever Regulation,” Crit. Rev. Immunol. 14:355-379), since the evidence indicates the hypocretins are arousal-related peptides. See Hagan et al. (1999), “Orexin A Activates Locus Coeruleus Cell Firing and Increases Arousal in the Rat,” Proc. Natl. Acad. Sci. USA 96:10911-10916 and Piper et al. (2000), “The Novel Brain Peptide, Orexin-A, Modulates the Sleep-Wake Cycle of Rats,” Euro. J. Neurosci. 12:726-730. Patients who undergo α-IFN therapy report excessive sleepiness. See Smedley et al. (1983), “Neurological Effects of Recombinant Human Interferon,” Br. J Med. 286:262-266. Furthermore, α-IFN expression has been reported to be enhanced during and after sleep deprivation. See Palmblad et al. (1976), “Stress Exposure and Immunological Response in Man: Interferon-Producing Capacity and Phagocytosis,” J. Psychosom. Res. 29:193-198. The effect of α-IFN is believed to be mediated, in part, by interleukin-1 (IL-1). See Krueger et al. (1994), supra. IL-1, well established as a somnogenic cytokine, is induced by α-IFN. See Gerrard et al. (1986), “Differential Effects of Interferon Alpha and Gamma on Interleukin-1 Secretion by Monocytes,” J. Immunol. 138:2535-2540.

Although the ISRE has been associated with the transcriptional activation of target genes (Kerr et al. (1991), “The Control of Interferon-Inducible Gene Expression,” FEBS Lett. 285:194-198), it is an inhibitor of transcription of the hypocretin gene. Activation by α-IFN can occur through binding of Stats (signal transducers and activators of transcription) 1 and 2 and a protein called p48 to the ISRE. See Levy (1995) “Interferon Induction of Gene Expression Through the Jak-Stat Pathway,” Semin. Virol. 6:181-190 and Bluyssen et al. (1996), “ISGF3γp48, A Specificity Switch for Interferon Activated Transcription Factors,” Cyt. Growth Fact. Rev. 7:11-17. P48 is a member of the IFN regulatory factor-1 (IRF-1) family of proteins. Although the seven-member Stat family of proteins plays highly specific functional roles, it is possible that Stat1 and/or Stat2 may also exhibit suppressor activity and, through this activity, inhibit expression of the preprohypocretin gene. In a recent study, Stat5 proteins were shown to suppress expression of the enzyme, 20-□-hydroxysteroid dehydrogenase (20□SDH), in the ovary. See Teglund et al. (1998), “Stat5a and Stat5b Proteins Have Essential and Nonessential or Redundant Roles in Cytokine Responses.” Cell 93:841-850. This finding is contrary to a previous report that had attributed suppression of 20□SDH to prolactin signaling. See Zhong et al. (1997), “Prolactin-Mediated Inhibition of 2-Alpha-Hydroxysteroid Dehydrogenase Gene Expression and the Tyrosine Kinase System,” Biochem. Biophys. Res. Commun. 135:587-592. Stat5b is known to down-regulate transcription of peroxisome proliferator-activated receptor alpha gene. See Zhou et al. (1999), “Stat5b Down-Regulates Peroxisome Proliferator-Activated Function Region-1 Trans-Activated Domain,” J. Biol. Chem. 274:29874-29882. In SY5Y cells, Stat1 or Stat2 may exert suppression of preprohypocretin gene expression either directly or indirectly through interaction with other regulatory proteins. Thus, Stats 1 and 2 may bind other members of the IRF-1 family that have repressor activity. See Mamane et al. (1999), supra. Alternatively, the inhibitory effect of α-IFN could be mediated through other families of regulatory proteins involved in the down-regulation of cytokine signaling, such as SOCS (suppressors of cytokine signaling). See Starr et al. (1997), “A Family of Cytokine-Inducible Inhibitors of Signaling,” Nature 387:917-921. SOCS-3 is expressed and inhibits the leptin receptor from signaling in the lateral hypothalamus where hypocretin cell bodies are located, as shown by Bjorbaek et al. (1998), “Identification of SOCS-3 as a Potential Mediator of Central Leptin Resistance,” Mol. Cell 1:619-625. Leptin is an adipocyte-derived hormone that has been implicated in appetite control. See Campfield et al. (1995), “Recombinant Mouse OB Protein: Evidence for a Peripheral Signal Linking Adiposity and Central Neural Networks,” Science 269:546-549; Halaas et al. (1996), “Weight Reducing Effects of the Plasma Protein Encoded by the Obese,” Science 269:543-546; and Pelleymounter et al. (1995), “Effects of Obese Gene Product on Body Weight Regulation in ob/ob Mice,” Science 269:540-543. As mentioned above, hypocretin/orexin peptides have been implicated in feeding behavior. See Sakurai et al. (1998), supra. Thus, the present invention demonstrates, among other things, the ability to modulate the hypocretin system of an individual through administration of a preprohypocretin-expression modulator.

What is claimed is:

1. A method for identifying a compound that modulates the hypocretin system comprising contacting a test compound to cells equipped with the 5′ flanking promoter of the preprohypocretin gene operably linked to a nucleic acid sequence and determining whether the test compound alters transcription of the nucleic acid sequence in the cells, wherein the test compound's ability to alter transcription is indicative of a compound that modulates the hypocretin system.

2. The method of claim 1, wherein the cells are naturally occurring preprohypocretin-expressing cells.

3. The method of claim 2, wherein the naturally occurring preprohypocretin cells are isolated from the posterior hypothalamus, bladder tissue, or tissue of the gastrointestinal tract.

4. The method of claim 1, wherein the cells are genetically manipulated to be equipped with the 5′ flanking promoter of the preprohypocretin gene.

5. The method of claim 4, wherein the genetically manipulated cells are selected from the group consisting of neuroblastoma cells, kidney cells, and ovary cells.

6. The method of claim 5, wherein the nucleic acid sequence codes for a known gene.

7. The method of claim 6, wherein alteration of transcription is evidenced by a change in expression of the gene when compared to expression of the gene without the compound.

8. The method of claim 7, wherein the compound enhances expression of the gene.

9. The method of claim 8, wherein the compound binds to the 5′-flanking promoter of the preprohypocretin gene.

10. The method of claim 7, wherein the compound decreases expression of the gene.

11. The method of claim 10, wherein the compound binds to the 5′-flanking promoter of the preprohypocretin gene.

12. The method of claim 1, which is carried out in vitro.

13. An isolated DNA fragment coding for the 5′ flanking promoter of the preprohypocretin gene.

14. The isolated DNA fragment of claim 13, comprising the nucleotide sequence of SEQ ID NO: 1.

15. An expression vector comprising the DNA fragment of claim 13.

16. The expression vector of claim 15, wherein the DNA fragment comprises the nucleotide sequence of SEQ ID NO: 1.

17. A host cell transformed with the expression vector of claim 15.

18. A compound that modulates the hypocretin system, wherein the compound is identified by the steps comprising contacting a preprohypocretin-expressing cell with the compound and determining whether the compound alters preprohypocretin expression in the preprohypocretin-expressing cell, wherein the compound's ability to alter preprohypocretin expression is indicative of a compound that modulates the hypocretin system.

19. A pharmaceutical composition comprising a therapeutically effective amount of the compound of claim 18.

20. The composition of claim 19, further comprising a pharmaceutically acceptable carrier.


SEQUENCE LISTING (9 sequences)

SEQ ID NO: 1 - DNA, 474 bp, Homo sapiens
gcagctaaggagcctttccatgaaggaagaaggtcctggagcctgacagtccccaggagcagcgacaagaagcaggggagggagaggactgctgctggctgctccaccccccacacacataatgtggggtctcgcgtctgcctctctcccgcccctaattagcagctgcctccctccatattgtcccaggccagcgcctcttttgtgctcccagattcctgggtgcaaggtggcctcattagtgcccggagaccgccccatctccagggagcagatagacagacaagggggtgatcaggggcacagtgatccaaccctggcctctgaacgccgcagcggccattccttgggcccagcctggagacggcccccctgcagcaggctaatcttagacttgcctttgtctggcctgggtgtggacgcaagtgcctgtcaattccccgccacctcagagcactataaaccccagacccc

SEQ ID NO: 2 - DNA, 24 bp, Artificial Sequence
  Description of Artificial Sequence Primer
ggggtctggggtttatagtgctct

SEQ ID NO: 3 - DNA, 24 bp, Artificial Sequence
  Description of Artificial Sequence Primer
gcagctaaggagcctttccatgaa

SEQ ID NO: 4 - DNA, 21 bp, Artificial Sequence
  Description of Artificial Sequence Primer
tccagggagcagatagacaga

SEQ ID NO: 5 - DNA, 21 bp, Artificial Sequence
  Description of Artificial Sequence Primer
gctaatcttagacttgccttt

SEQ ID NO: 6 - DNA, 30 bp, Artificial Sequence
  Description of Artificial Sequence Primer
ctacgcgctcggggctaagattagcctgct

SEQ ID NO: 7 - DNA, 28 bp, Artificial Sequence
  Description of Artificial Sequence Primer
gccccgagcgcgtagggcctgggtgtgg

SEQ ID NO: 8 - DNA, 14 bp, Artificial Sequence
  Description of Artificial Sequence Synthetic oligonucleotide
acttgcctttgtct

SEQ ID NO: 9 - DNA, 14 bp, Artificial Sequence
  Description of Artificial Sequence Synthetic oligonucleotide
ccccgagcgcgtag